(19) United States
(12) Patent Application Publication
(10) Pub. No.: US 2008/0132787 A1
(43) Pub. Date: Jun. 5, 2008

(54) Radiotherapeutic Apparatus

(75) Inventor: Miikka Putaala, Kirkkonummi (FI)

Correspondence Address: LAHIVE & COCKFIELD, LLP, ONE POST OFFICE SQUARE, BOSTON, MA, 02109-2127, US

(73) Assignee: Elekta AB (publ), Sockholm (SE)

(21) Appl. No.: 11/794,064

(22) PCT Filed: Dec. 15, 2005
(86) PCT No.: PCT/EP05/13489

§ 371 Date: Jun. 22, 2007

(30) Foreign Application Priority Data
Dec. 22, 2004 (GB) 0428023.6

(51) Int. Cl. A61B 8/00; A61N 5/00

(52) U.S. Cl. 600/439; 600/1

(57) ABSTRACT

The co-registration of a volumetric ultrasound acquisition of the volume of interest with a magnetic resonance image allows the former to providing the basis for calculation of the dose distribution and the latter to provide the basis for target delineation and isocenter placement. The present invention therefore provides an apparatus for planning the radiotherapeutic treatment of a volume of tissue, comprising a sonographic apparatus for acquiring acoustic data relating to the volume, a means for reconstruction of an internal structure of the volume on the basis of the acoustic data, and a means for classification of the material type within that internal structure to one or more tissue types. A three-dimensional motion-tracking device for the sonographic device will assist. The sonographic device will typically be an ultrasound probe. The tissue type data can be passed to a dosage calculation means for determination of radiation dosage. The greater accuracy of the three-dimensional electron density data that can then be used given that the internal variation of tissue type is known will mean that the treatment can be modelled more accurately and, therefore, a more optimal treatment can be determined using, for instance, magnetic resonance imaging (MRI) alone.

### FIELD OF THE INVENTION

The present invention relates to a radiotherapeutic apparatus. It seeks to address issues relating to the planning of radiotherapeutic treatment.

### BACKGROUND ART

Most current dose calculation algorithms for stereotactic radiosurgery or radiation therapy either make use of electron-density values derived from computerized tomography (CT) investigation, or they assume that the body consists of homogenous material, such as water. This is then used as the basis for estimating attenuation of the radiation that will be used to treat the lesion. This may be x-radiation or other biologically effective radiation.

These approaches are cumbersome, especially in the context of stereotactic radiosurgery, which normally does not require the acquisition of computerized tomograms for dose calculation. While CT scanning is nearly ideal in terms of the accuracy of tissue density classification, the acquisition of computerized tomograms for the purposes of treatment planning is time consuming, results in the delivery of extraneous radiation dose to the patient, and is costly. Approaches based on an assumption of tissue homogeneity are, in general, vulnerable to inhomogeneities in the volume of interest such as air cavities, and thus are unreliable and inaccurate in the vicinity of (for example) the patient's skull and lungs.

Some attempts have been made based on magnetic resonance imaging, but these are limited due to the erratic nature of automatic segmentation methods and the amount of manual labor required to segment the volume of interest manually.

### SUMMARY OF THE INVENTION

The present invention attempts to address the above problems by means of three-dimensional sonography. The approach employs the co-registration of a volumetric ultrasound acquisition of the volume of interest with magnetic resonance imaging, the former providing the basis for calculation of the dose distribution and the latter the basis for target delineation and isocenter placement.

The present invention therefore provides an apparatus for planning the radiotherapeutic treatment of a volume of tissue, comprising a sonographic apparatus for acquiring acoustic data relating to the volume, a means for reconstruction of an internal structure of the volume on the basis of the acoustic data, and a means for classification of the material type within that internal structure to one or more tissue types.

To assist with the automation of the classification of tissue type, the apparatus preferably includes a three-dimensional motion-tracking device for locating the sonographic device. Ideally, this will locate the sonographic device with respect to both position and orientation.

The sonographic device will typically be an ultrasound probe.

As noted above, the apparatus can also comprise a further scanner for obtaining three-dimensional volume data regarding the volume. This will ideally use investigative means other than x-rays in order to minimise the dosage delivered to the patient. A magnetic resonance imaging scanner is ideal for the purpose. There will then preferably be a means for registration of the three-dimensional outputs of the reconstruction means and the further scanner, thereby to produce tissue type data and three-dimensional structure data in a region of interest within the volume.

The tissue type data can be passed to a dosage calculation means for determination of radiation dosage. The greater accuracy of the three-dimensional electron density data that can then be used given that the internal variation of tissue type is known will mean that the treatment can be modelled more accurately and, therefore, a more optimal treatment can be determined.

Thus, the invention further provides a radiotherapeutic apparatus comprising a treatment planning device which receives the tissue classified volume data and prepares a dosage plan taking into account the radiation attenuation of the tissue types detected, a radiation source, and a control means for the radiation source, the control means being adapted to deliver a treatment according to the dosage plan determined by the dosage calculation means.

### BRIEF DESCRIPTION OF THE DRAWINGS

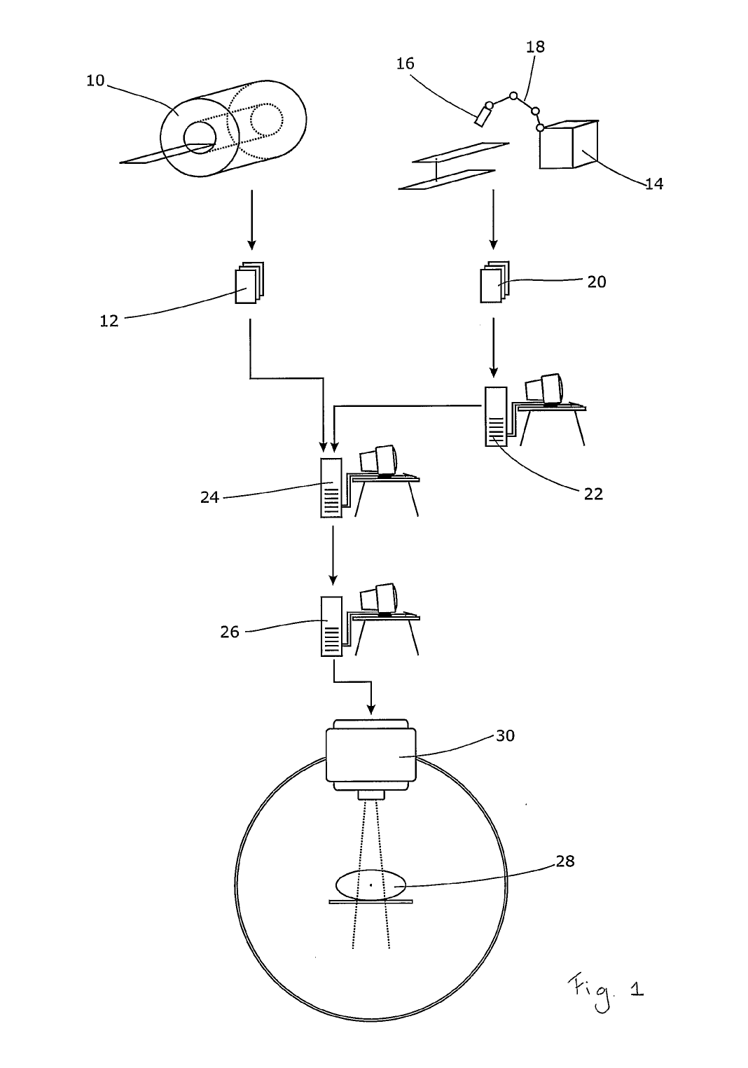

An embodiment of the present invention will now be described by way of example, with reference to the accompanying figures in which FIG. 1 schematically illustrates the present invention.

### DETAILED DESCRIPTION OF THE EMBODIMENTS

In technical terms, the solution could comprise (a) a three-dimensional, real-time motion tracking system; (b) an ultrasound device; (c) software for reconstruction of a three-dimensional ultrasound acquisition; (d) software for tissue classification; and (e) software for co-registering the classified three-dimensional sonographic acquisition with magnetic resonance images. The real-time motion tracking system provides the positions and orientations of the relevant part of the subject's body and of the ultrasound probe. The ultrasound device is used to interactively scan the volume of interest, the result of which is a volumetric reconstruction of the sonography. The volumetric reconstruction is subsequently classified such that density values comparable to those available from computerized tomography result. The classified volumetric reconstruction is finally co-registered with a magnetic resonance image set and used in place of a computerized tomogram as the basis of the dose calculation.

The advantages of the approach presented herein within are that it eliminates the need for acquisition of computerized tomography even with more advanced dose calculation algorithms, such as ray tracing or convolution-based approaches. Consequently, the approach reduces the overall radiation exposure to the patient, provides cost savings due to the relative inexpensiveness of the sonographic equipment, and permits time-efficient acquisition of density data within the radiosurgical or radiation therapy suite.

In terms of disadvantages, the accuracy of the present approach is, to a degree, operator dependent and necessitates the availability of an acoustic window (the ultrasound probe must be placed against acoustically conductive tissue). Furthermore, the classification of tissue is still somewhat unproven, although successful attempts have been made to convert ultrasound velocity data to density values comparable to those available from computerized tomography. For instance, Saulgois and Pontaga (1998) used multi factor linear and non linear correlation and regression analyses to find the conversion function, while Kutay et al. (2003) constructed a model of ultrasonic echoes in an attempt to classify breast tissue. Further, Feleppa et al. (2002) used ultrasonic spectrum analysis and neural networks to accurately classify tissue in the context of brachytherapy of porstate cancer. Collectively, these authors were able to accomplish reasonably reliable and accurate classifications, demonstrating the viability of the technique even in the clinical setting. Finally, the accuracy of the registration of three-dimensional reconstructions of sonograms with magnetic resonance imaging is limited in the presence of moving organs, although successful attempts have been made, for instance, by Penney et al. (2004) to overcome such limitations.

The newness of the present approach is in the use of three-dimensional, density-classified sonography in the context of radiosurgery or radiation therapy. Prior art exists, separately, in the areas of ultrasound-based tissue classification and of three-dimensional sonography but appears non-existing in the context of radiosurgery or radiation therapy.

Thus, referring to FIG. 1, a patient is placed in an investigative scanner such as an MRI scanner 10. From this, a three-dimensional data set 12 is obtained. The nature of an MRI scanner is such that this data set will contain information as to the internal structure of the patient.

The patient is also investigated via a sonographic means such as the ultrasound device shown at 14. The sensor head 16 is manipulated via a three-dimensional locating device 18 consisting of an articulated arm whose articulations are quantified, such as by potentiometers. This allows the position and (preferably) the orientation of the sensor head 16 to be determined. As a result, the ultrasound scanner 14 is able to produce a data set 20 containing information as to the edges and densities within the patient.

Other means of locating the sonographic means exist, such as systems based on magnetic fields or stereoscopic infrared cameras. Particularly, those approaches based on a magnetic field appear equally or more preferable due to their ability to provide high accuracy without the physical limitations imposed by an articulated arm.

The data set 20 can be passed to a computing means 22 to classify areas within the data set according to the tissue type. This can be at a gross level, such as tissue vs non-tissue, or can be by way of classification into finer categories such as bone, aqueous fluid, cavity or other tissue. In either case, the density information obtained from the ultrasound scan can be compared with known values to allow classification.

The data set 20 after classification and the MRI data set 12 can then be registered by a computing means 24, so that they are placed in the same frame of reference and locations in the images can be compared directly. This means that the tissue type data obtained from the ultrasound investigation can be correlated with the structure data obtained from the MRI investigation. This combined data set is then passed to a treatment planning means 26 which uses the tissue type data and known attenuation rates to produce a proposed radiotherapeutic treatment. After validation and/or approval by the physician, the patient 28 can then be treated by the radiotherapeutic apparatus 30.

It will of course be understood that many variations may be made to the above-described embodiment without departing from the scope of the present invention.

### REFERENCES

Feleppa, E. J., Ennis, R. D., Schiff, P. B., Wuu, C-S., Kalisz, A., Ketterling, J., Urban, S., Liu, T., Fair, W. R., Porter, C. R., and Gillespie, 3. R. (2002) Ultrasonic spectrum-analysis and neural-network classification as a basis for ultrasonic imaging to target brachytherapy or prostate cancer. Brachyteraphy, Vol. 1 (2002), pp. 48-53.
Kutay, M. A., Petropulu, A. P., and Piccoli, C. W. (2003) Breast tissue characterization based on modelling of ultrasonic echoes using the power-law shot noise model. Pattern Recognition Letters, Vol. 24 (2003), pp. 741-56.
Penney, G. P., Blackall, 3. M., Hamady, M. S., Sabharwal, T., Adam, A., and Hawkes, D. J. (2004) Registration of freehand 3D ulstrasound and magnetic resonance liver images. Medical Image Analysis, Vol. 8 (2004), pp. 81-91.
Saulgois J. and Pontaga, I. (1998) Relationship between X-ray density, calcium content and ultrasound propagation in the human tibia. Working paper. Riga Technical University.

1. Apparatus for planning the radiotherapeutic treatment of a volume of tissue, comprising;
a sonographic apparatus for acquiring acoustic data relating to the volume,
a means for reconstruction of an internal structure of the volume on the basis of the acoustic data;
a means for classification of the material type within that internal structure to one or more tissue types.

2. Apparatus for planning the radiotherapeutic treatment of a volume according to claim 1, further including a three-dimensional motion tracking device for locating the sonographic device.

3. Apparatus for planning the radiotherapeutic treatment of a volume according to claim 2, in which the three dimensional motion tracking device locates the sonographic device with respect to both position and orientation.

4. Apparatus for planning the radiotherapeutic treatment of a volume according to claim 1 in which the sonographic device is an ultrasound probe.

5. Apparatus for planning the radiotherapeutic treatment of a volume according to claim 1 comprising a further scanner for obtaining three-dimensional volume data regarding the volume.

6. Apparatus for planning the radiotherapeutic treatment of a volume according to claim 5 in which the further scanner uses investigative means other than x-rays.

7. Apparatus for planning the radiotherapeutic treatment of a volume according to claim 5 in which the further scanner is a magnetic resonance imaging scanner.

8. Apparatus for planning the radiotherapeutic treatment of a volume according to claim 1 comprising means for registration of the three-dimensional outputs of the reconstruction means and the further scanner thereby to produce tissue type data in a region of interest within the volume.

9. Apparatus for planning the radiotherapeutic treatment of a volume according to claim 8, in which the tissue type data is passed to a dosage calculation means for determination of radiation dosage.

10. Radiotherapeutic apparatus comprising;
a treatment planning device according to claim 1 which receives the issue classified volume data and prepares a dosage plan taking into account the radiation attenuation of the tissue types detected
a radiation source, and
a control means for the radiation source, the control means being adapted to deliver a treatment according to the dosage plan determined by the dosage calculation means.
